SEQUENCE LISTING (28 sequences)

SEQ ID NO: 1 - DNA, 14 bp, Artificial Sequence
  blocking double stranded nucleic acid complex for use in nucleic acid amplification
gccaatcctacgcc

SEQ ID NO: 2 - DNA, 14 bp, Artificial Sequence
  blocking double stranded nucleic acid complex for use in nucleic acid amplification
gccaatcctacgcc

SEQ ID NO: 3 - DNA, 14 bp, Artificial Sequence
  blocking double stranded nucleic acid complex for use in nucleic acid amplification
gccaatcctacgcc

SEQ ID NO: 4 - DNA, 12 bp, Artificial Sequence
  blocking double stranded nucleic acid complex for use in nucleic acid amplification
gccggccaatgt

SEQ ID NO: 5 - DNA, 15 bp, Artificial Sequence
  blocking double stranded nucleic acid complex for use in nucleic acid amplification
cctgacaatgccgcg

SEQ ID NO: 6 - DNA, 15 bp, Artificial Sequence
  blocking double stranded nucleic acid complex for use in nucleic acid amplification
cctgacaatgccgcg

SEQ ID NO: 7 - DNA, 18 bp, Artificial Sequence
  blocking double stranded nucleic acid complex for use in nucleic acid amplification
agcggataacaatatcac

SEQ ID NO: 8 - DNA, 9 bp, Artificial Sequence
  blocking double stranded nucleic acid complex for use in nucleic acid amplification
gccaatcat

SEQ ID NO: 9 - DNA, 10 bp, Artificial Sequence
  blocking double stranded nucleic acid complex for use in nucleic acid amplification
gccaatccta

SEQ ID NO: 10 - DNA, 11 bp, Artificial Sequence
  blocking double stranded nucleic acid complex for use in nucleic acid amplification
gccaatcctac

SEQ ID NO: 11 - DNA, 12 bp, Artificial Sequence
  blocking double stranded nucleic acid complex for use in nucleic acid amplification
gccaatcctacg

SEQ ID NO: 12 - DNA, 13 bp, Artificial Sequence
  blocking double stranded nucleic acid complex for use in nucleic acid amplification
gccaatcctacgc

SEQ ID NO: 13 - DNA, 17 bp, Artificial Sequence
  blocking double stranded nucleic acid complex for use in nucleic acid amplification
gccaatcctacgcctcc

SEQ ID NO: 14 - DNA, 19 bp, Artificial Sequence
  blocking double stranded nucleic acid complex for use in nucleic acid amplification
gccaatcctacgcctccgt

SEQ ID NO: 15 - DNA, 27 bp, Artificial Sequence
  blocking double stranded nucleic acid complex for use in nucleic acid amplification
gccaatcctacgcctccgtgacgatcc

SEQ ID NO: 16 - DNA, 31 bp, Artificial Sequence
  blocking double stranded nucleic acid complex for use in nucleic acid amplification
gccaatcctacgcctccgtgacgatccgctc

SEQ ID NO: 17 - DNA, 16 bp, Artificial Sequence
  Primer
ctggctgacattttcg

SEQ ID NO: 18 - DNA, 18 bp, Artificial Sequence
  Primer
tatcgacatttctgcacc

SEQ ID NO: 19 - DNA, 34 bp, Artificial Sequence
  Primer
gaagtcaacaaaaaggagctggctgacattttcg

SEQ ID NO: 20 - DNA, 32 bp, Artificial Sequence
  Primer
cagcagatacgggatatcgacatttctgcacc

SEQ ID NO: 21 - DNA, 23 bp, Artificial Sequence
  Primer
aacaatttcacacaggaacagct

SEQ ID NO: 22 - DNA, 19 bp, Artificial Sequence
  Primer
gttttcccagtcacgacgt

SEQ ID NO: 23 - DNA, 22 bp, Artificial Sequence
  Primer
aacaatttcacacagcaacagc

SEQ ID NO: 24 - DNA, 18 bp, Artificial Sequence
  Primer
gttttcccagtcacgacg

SEQ ID NO: 25 - DNA, 22 bp, Artificial Sequence
  Primer
aaggagctggctgacattttcg

SEQ ID NO: 26 - DNA, 23 bp, Artificial Sequence
  Primer
cgggatatcgacatttctgcacc

SEQ ID NO: 27 - DNA, 20 bp, Artificial Sequence
  Primer
agagatggccacggctgctt

SEQ ID NO: 28 - DNA, 20 bp, Artificial Sequence
  Primer
atttgcggtggacgatggag

(19) United States
(12) Patent Application Publication
(10) Pub. No.: US 2011/0111462 A1
(43) Pub. Date: May 12, 2011

(54) Composition and Method for Synthesizing a Deoxyribonucleotide Chain Using a Double Stranded Nucleic Acid Complex with a Thermostable Polymerase

(76) Inventors: Stephen Picone, Beverly, MA (US); Christopher Benoit, South Hamilton, MA (US); Bilyana Koleva, Boston, MA (US)

(21) Appl. No.: 12/940,307

(22) Filed: Nov. 5, 2010

Related U.S. Application Data
(60) Provisional application No. 61/258,684, filed on Nov. 6, 2009.

(51) Int. Cl. C12P 19/34; C07H 21/00; C12N 9/00

(52) U.S. Cl. 435/91.2; 536/23.1; 435/183

(57) ABSTRACT

The present invention relates to the field of molecular biology, and more particular, to a nucleic acid construct for use in amplification processes. More precisely, the invention enhances the specificity of amplification of nucleic acids by means of a double stranded oligonucleotide modified with a molecule having the ability to prevent extension of the double stranded nucleic acid.

### RELATED APPLICATION

This application claims the benefit of U.S. Provisional Application No. 61/258,684, filed Nov. 6, 2009, entitled “Composition and Method for Synthesizing a Deoxyribonucleotide Chain Using a Double Stranded Nucleic Acid Complex with a Thermostable Polymerase” by Stephen Picone, et al.

The entire teachings of the above application are incorporated herein by reference.

### BACKGROUND OF THE INVENTION

DNA polymerases are enzymes that catalyze the polymerization of deoxyribonucleotides into strands of nucleic acid. Polymerases are used in various DNA techniques including PCR amplification, namely a process to copy or amplify DNA strands. A significant problem with certain PCR methods is the generation of non-specific amplification products (e.g., the creation of unwanted DNA strands). In many cases, this is due to non-specific oligonucleotide priming and production of non-target oligonucleotides of side-reactions, such as mispriming of a background DNA and/or primer oligomerization and subsequent primer extension event prior to the actual thermocycling procedure itself. This often occurs because thermostable DNA polymerases are moderately active at ambient temperature.

In order to minimize this problem, a method known as “hot start” PCR can be performed. In hot start PCR, one component essential for the amplification reaction is either separated from the reaction mixture or kept in an inactive state until the temperature of the reaction mixture is being raised for the first time. Since the polymerase cannot function under these conditions, there is less primer elongation during the period when the primers can bind non-specifically. In order to achieve this effect, several methods have been applied: Physical Separation of the DNA polymerase (e.g., using a barrier of solid wax to separate the DNA polymerase from the reaction mixture), chemical modification of DNA polymerase (e.g., DNA polymerase is reversibly inactivated, polymerase DNA antibodies (e.g., antibodies bind at ambient temperatures and disassociate at higher temperatures during amplification), DNA polymerase inhibition by nucleic acid additives, aptamers (e.g., single strand form of nucleotides that form loops, pseudoknots, and complicated tertiary structures that act like an antibody), blocked primers, and others. Several of these methods are either inconvenient or do not work as well as desired to minimize non-specific amplification.

Accordingly, a need exists for a unique and alternative composition and method for amplification reactions, which allows for an inhibition of non-specific priming and primer extension not only prior to the amplification process itself but also during the thermocycling process. More specifically, a need exists for an alternative and improved composition and method for hot start PCR.

### SUMMARY OF THE INVENTION

The present invention relates to a blocking double stranded nucleic acid complex (DSC) for use in nucleic acid amplification. In an embodiment, the complex includes an isolated double stranded nucleic acid molecule that has a first nucleic acid strand having a first sequence comprising between about 9 to about 40 nucleic acid bases, and a nucleic acid second strand having a second sequence comprising between about 9 to about 40 nucleic acid bases that are complementary to the first sequence, wherein the first nucleic acid strand and the second nucleic acid strand each having a 3′ end and a 5′ end, and wherein the double stranded nucleic acid molecule has a percentage of cytosine (C) and guanine (G) in a range between about 50% and about 70%. The double stranded nucleic acid complex also includes a blocking molecule, wherein the blocking molecule is covalently bonded to the 3′ end or the 5′ end of the first nucleic acid strand, the second nucleic acid strand, or both. In an aspect, the double stranded nucleic acid molecule (e.g., DNA, or RNA) has a melting temperature in a range between about 25° C. and about 90° C. In an embodiment, the DSC of the present invention further includes the introduction of uracil bases. The addition of one or more uracil bases to the DSC further reduces polymerase activity at room temperature when using a DNA polymerase from a species of an Archaebacteria. In particular, the first sequence or the second sequence of the DSC further comprises one or more uracil bases.

Examples of blocking molecules include deoxythymidine, dideoxynucleotides, 3′ phosphorylation, hexanediol, spacer molecules, 1′2′-dideoxyribose, 2′-0-Methyl RNA, and/or Locked Nucleic Acids (LNAs). In an embodiment, the blocking molecule has the following structure:

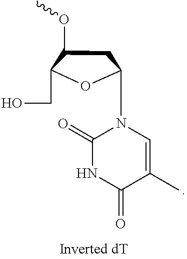

The present invention also pertains to a blocking double stranded nucleic acid complex for use in nucleic acid amplification; wherein the complex includes an isolated double stranded nucleic acid molecule that has a first nucleic acid strand having a first sequence comprising between about 9 to about 40 nucleic acid bases, and a nucleic acid second strand having a second sequence comprising between about 9 to about 40 nucleic acid bases that are complementary to the first sequence, wherein the first nucleic acid strand and the second nucleic acid strand each having a 3′ end and a 5′ end, and the double stranded nucleic acid molecule has a melting temperature in a range between about 25° C. and about 90° C. This embodiment includes a blocking molecule that is covalently bonded to the 3′ end or the 5′ end of the first nucleic acid strand, the second nucleic acid strand, or both. In an aspect, the first sequence or the second sequence further comprises one or more uracil bases.

In yet another embodiment, the blocking double stranded nucleic acid complex of the present invention has a complex that includes an isolated double stranded nucleic acid molecule that has a first nucleic acid strand having a first nucleic acid sequence greater than or equal to about 70% identity with one of the following sequences: SEQ ID NO: 1, 2, 3, 4, 5, 6, 7, 8, 9, 10, 11, 12, 13, 14, 15, 16, or combination thereof; a complement of SEQ ID NO: 1, 2, 3, 4, 5, 6, 7, 8, 9, 10, 11, 12, 13, 14, 15, 16, or combination thereof; or a sequence that hybridizes to SEQ ID NO: 1, 2, 3, 4, 5, 6, 7, 8, 9, 10, 11, 12, 13, 14, 15, 16, or combination thereof. The complex further includes a second nucleic acid strand having a second sequence comprising between about 9 to about 40 nucleic acid bases that are complementary to the first nucleic acid sequence, wherein the first nucleic acid strand and the second nucleic acid strand each having a 3′ end and a 5′ end, wherein the double stranded nucleic acid molecule has a melting temperature in a range between about 25° C. and about 90° C. The complex also has a blocking molecule that is covalently bonded to the 3′ end or the 5′ end of the first nucleic acid strand, the second nucleic acid strand, or both. In an aspect, the 3′ end of the first and second nucleic acid strand comprises the blocking molecule, and when the blocking double stranded nucleic acid complex interacts with a nucleic acid polymerase, the non-specific amplification products are thereby reduced. The complex has a melting temperature in a preferred embodiment of about 48.9° C.

The present invention also includes compositions for nucleic acid amplification. In certain embodiments, the composition includes a buffer, the blocking double stranded nucleic acid complex described herein, and a thermostable polymerase. The polymerase can be a DNA polymerase such as Taq DNA polymerase; BST DNA Polymerase; PFU DNA polymerase; Klenow DNA polymerase; T7 DNA polymerase; T4 DNA polymerase; Phi29 DNA polymerase; or RB69 DNA polymerase. The range of concentration can be between e.g., about 2 μM nucleic acid complex to every 5,000 U/mL of polymerase and 2 mM nucleic acid complex for every 5,000 U/mL of polymerase. The buffer can be a TRIS buffer, MOPS, or a HEPES buffer. Methods of amplifying a target nucleic acid molecule are further encompassed by the present invention. The method includes contacting the target nucleic acid molecule with a DNA polymerase and the double stranded nucleic acid complex, as described herein, wherein the double stranded nucleic acid complex binds to the DNA polymerase, at a temperature, ranging from about 25° C. to about 90° C. The production of one or more non-specific amplification products or secondary products is reduced, as compared to that not contacted with the double stranded nucleic acid complex. Polymerase activity at room temperature (e.g., between about 20° C. and 25° C.) is reduced in a range between about 50% and about 90% (e.g., about a 50%, 60%, 70%, 80% or 90% reduction), as compared to polymerase activity for target nucleic acid molecules not contacted with the double stranded nucleic acid complex. Additionally, the methods of the present invention, in an aspect, provide a greater yield. In a particular embodiment, the amount of amplified target nucleic acid molecules is increased in a range between about 2× and about 20× (e.g., a 2×, 3×, 4×, 5×, 6×, 7×, 8×, 9×, 10×, 15× or 20× increase), as compared to the amount obtained for target nucleic acid molecules not contacted with the double stranded nucleic acid complex. In yet another embodiment in which the method is utilizing a DNA polymerase from a species of an Archaebacteria and a DSC having one or more uracil bases, polymerase activity at room temperature is also reduced, as described herein.

Additional methods embodied by the present invention include amplifying a target nucleic acid molecule by mixing a buffer, the target nucleic acid molecule, one or more primers, a DNA polymerase, a supply of adenine, guanine, cytosine and thymine, and the double stranded nucleic acid complex described herein. The steps further include allowing for amplification of the target nucleic acid molecule by increasing the temperature in one or more cycles, wherein the temperature ranges between about 25° C. to about 90° C. The method allows for the production of one or more non-specific amplification products or secondary products to be reduced as compared to that not contacted with the double stranded nucleic acid complex. In an embodiment, polymerase activity at room temperature is reduced and/or an increase in yield is obtained, as further described herein.

The present invention further includes kits for nucleic acid amplification. Such a kit or system includes the blocking double stranded nucleic acid complex described herein, and a polymerase. The polymerase is a DNA polymerase can be e.g., Taq DNA polymerase; BST DNA Polymerase; PFU DNA polymerase; Klenow DNA polymerase; T7 DNA polymerase; T4 DNA polymerase; Phi29 DNA polymerase; or RB69 DNA polymerase.

Advantageously, the claimed invention provides compositions and methods for improving the hot start PCR amplification process. Specifically, the present invention provides a composition that inhibits non-specific priming and primer extension prior to and during the amplification process. Additionally, the present invention surprisingly allows for PCR reactions to occur without a significant amount of non-specific amplification product, and provides for an improved composition for performing PCR.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 depicts the amplification of a 1.1 kb region of pUC19 plasmid. Lanes 1 and 2 include reactions with 3′ end capped primers in PCR buffer I and II, respectively, showing that an inhibitory molecule incorporated at the 3′ end of an oligonucleotide effectively prevents the extension by a DNA polymerase. Lanes 3 and 4 depict reactions with non-modified primers resulting in amplification product. Lane M depicts DNA standard marker.

FIG. 2 depicts PCR product amplification of a 1.1 kb region of the pUC19 plasmid in the presence and absence of double stranded nucleic acid molecule with a blocking agent that has the same sequence as the reaction primers. Lanes 1 and 2 include reactions performed in PCR buffer I, lanes 3 and 4 were carried out in PCR buffer II. Lane M depicts DNA standard marker.

FIG. 3 illustrates the PCR amplification in the presence of DSC molecules. All reactions contain 1 ng of E. coli genomic DNA as a competing foreign DNA. Odd number lanes contain Lambda template DNA (except lane 11), whereas even number lanes do not contain template DNA. Reaction 11 is a negative control having buffer I, DNTPs but with no DSC molecule, no template, and no enzyme. Lane M depicts DNA standard marker.

FIG. 4 depicts the comparison in amplification of a 1.9 kb region from Lambda DNA. Lanes 1-4 contain Taq (Enzymatics, Inc. (Beverly, Mass.) and Taq-B DNA polymerase (− and + stabilizer in storage buffer) in the presence of DSC molecules. Lanes 5-7 contain commercially available hot start DNA polymerases. All PCR reactions were performed in the presence of Lambda DNA and 1 ng of contaminating E. coli genomic DNA. Lane M depicts DNA standard marker.

FIGS. 5A and B emphasize the amplification of a 1.9 kb region of Lambda DNA in the presence of DSC molecule. PCR amplification was carried out in A with forward 5′CTGGCTGACATTTTCG-3′ (SEQ ID NO: 17) and reverse 5′TATCGACATTTCTGCACC-3′(SEQ ID NO: 18) primers; in B with forward 5′ GAAGTCAACAAAAAGGAGCTGGCTGACATTTTCG-3′ (SEQ ID NO: 19) and reverse 5′CAGCAGATACGGGATATCGACATTTCTGCACC-3′ (SEQ ID NO: 20) primers. PCR amplification was performed with 0.523 pg of Lambda DNA and 1 ng of E. coli genomic DNA. Reaction were performed in duplicates (lane 1 and 2, 3 and 4 in FIG. 5A; lane 1 and 3, 2 and 4 in FIG. 5B).

FIG. 6 depicts the amplification of a 653-bp fragment of the β-actin gene of human placental DNA in the presence of DSC molecules with varying melting Temperatures (lanes 1-10) and inhibitory blocking molecules (lanes 15-17).

FIGS. 7A and B depict the amplification of a 100-bp product from DNA B using oligonucleotide mix B. Lanes 1-3 show the PCR amplification in the presence of the different DSC molecules. Lane 4 shows the results of amplification in the absence of the DSC molecule of the invention. Amplification in lane 5 is executed with a commercially available chemically modified hot start Taq polymerase. Lane M depicts DNA standard marker. All lanes contain 1000 copies of DNA B. The final concentration of DSC molecule in each reaction is 0.4 μM. The PCR amplification in FIG. 7A was performed immediately after set-up with no pre-incubation at bench top. The PCR amplification in FIG. 7B was performed after 24 hour incubation at ambient Temperature of 23° C.

FIG. 8 shows the amplification of a 100-bp product from DNA B using oligonucleotide mix B. Lanes 1-4 show the PCR amplification in the presence of the different DSC molecules. Lane 5 shows the results of amplification in the absence of the DSC molecule of the invention. Amplification in lane 6 is executed with a commercially available chemically modified hot start Taq polymerase. Lane M depicts DNA standard marker. All lanes contain 5 copies of DNA B. The final concentration of DSC molecule in lanes 1 and 3 is 0.4 μM whereas the final concentration of DSC in lanes 2 and 4 is 4 μM. The PCR amplification was performed immediately after set-up with no pre-incubation at bench top.

FIG. 9 illustrates the amplification of a 100-bp product from DNA B using oligonucleotide mix B. Lanes 1-5 show the PCR amplification in the presence of the different DSC molecules. Lane 6 shows the results of amplification in the absence of the DSC molecule of the invention. Amplification in lane 7 is executed with a commercially available chemically modified hot start Taq polymerase. Lane M depicts DNA standard marker. All lanes contain 5 copies of DNA B. The final concentration of DSC molecule in lanes 1-5 is 4 μM. The PCR amplification was performed after 24 hour incubation at ambient Temperature of 23° C.

FIG. 10A-C depict the amplification of a 100-120-bp product from DNA A, B, and C using oligonucleotide mix A, B, and C respectively. Lanes 1-5 show the PCR amplification in the presence of the different DSC molecules. Lane 6 shows the results of amplification in the absence of the DSC molecule of the invention. Amplification in lane 7 is executed with a commercially available chemically modified hot start Taq polymerase. Lane M depicts DNA standard marker. All PCR amplification reactions contain 5 copies of DNA B. The final concentration of DSC molecule in each reaction is 4 μM. The PCR amplification was performed immediately after set-up with no pre-incubation at bench top.

FIG. 11A-C depict the amplification of a 100-bp product from DNA A, B, and C using oligonucleotide mix A, B, and C respectively. Lanes 1-5 show the PCR amplification in the presence of the different DSC molecules. Lane 6 shows the results of amplification in the absence of the DSC molecule of the invention. Amplification in lane 7 is executed with a commercially available chemically modified hot start Taq polymerase. Lane M depicts DNA standard marker. All lanes contain 5 copies of DNA B. The final concentration of DSC molecule in each reaction is 4 μM. The PCR amplification in FIG. 7B was performed after 24 hour incubation at ambient Temperature of 23° C.

FIG. 12 depicts the real-time PCR analysis of the formation of 100-bp product from DNA C using detection by CY5 fluorescent dye. Reactions, which contained from 1280-5 copies of DNA C were performed in quadruplicate. Average Ct values±Standard Deviation are shown for each copy level. Standard deviation above 0.6 is bolded. The overall PCR efficiency along with R-squared value for the equation line is also shown. Results are depicted by hatched markings.

FIG. 13 depicts the real-time PCR analysis of the formation of 100-bp product from human placental DNA using HBB2 oligo mix. Reactions, which contained from 1280-5 copies of DNA C were performed in quadruplicate. Average Ct values±Standard Deviation are shown for each copy level. Standard deviations above 0.6 are bolded. The overall PCR efficiency along with R-squared value for the equation line is also shown. Results are depicted by hatched markings.

FIGS. 14 A and B illustrate the real-time PCR analysis of the formation of 100-bp product from DNA B using detection by HEX fluorescent dye. Reactions, which contained 1000, 100, and 10 copies of DNA B were performed in quadruplicate. Average Ct values±Standard Deviation are shown for each copy level. FIG. 14A represents the amplification of product in 25 μL reaction with 2.5 U of Taq-B and 0.4 μM final concentration of DSC1. FIG. 14B shows the amplification curves of product in 50 μL reaction with 2.5 U of Taq-B and 0.2 μM final concentration of DSC1.

FIG. 15A-C illustrate the real-time PCR analysis of the formation of 100-bp product from DNA B using detection by HEX fluorescent dye. Reactions, which contained 1000, 100, and 10 copies of DNA B were performed in quadruplicate. The final concentration of DSC5 molecule in reaction was noted from 1×-20×, where 1× was 0.2 μM and 20× is 4 μM. DSC1 at 1×, 0.2 μM final concentration in the reaction was represented. Taq-B with no DSC molecule and Fast Start was shown as well. FIG. 15A depicts the average Ct values for each copy level in each category. The overall PCR efficiencies are shown. FIG. 15B illustrates the amplification curves for each Taq-B DSC combination along with Taq-B and FastStart alone. FIG. 15C shows the final amplitude for each copy level in each category. Results are depicted by hatched markings.

### DETAILED DESCRIPTION OF THE INVENTION

A description of preferred embodiments of the invention follows.

The present invention relates to a nucleic acid complex for use in an amplification reaction, methods of using the nucleic acid complex, a buffer containing a polymerase and a nucleic acid complex, as well as a kit containing a nucleic acid complex and a polymerase. As described herein, the present invention includes nucleic acid complexes which improve amplification reactions. The nucleic acid complex is a double stranded oligonucleotide comprising a blocking molecule. The nucleic acid complex of the invention is also known as a Double Stranded Complex (DSC).

In particular, the present invention uses a nucleic acid complex, made of a short, double stranded oligonucleotide covalently attached to a blocking molecule on the terminal end of each strand. In other embodiments, the blocking molecule can be interspersed or attached to any portion (e.g., a middle portion) of the nucleic acid complex. The nucleic acid complex binds to the polymerase and enhances performance of an amplification reaction. The nucleic acid ligand is carefully designed to improve the performance of the amplification reaction, while itself not able to act as a primer for the amplification of the target sequence or another non-specific sequence in the reaction.

Also detailed experiments, described in the Exemplification Section, have been performed using a number of combinations of the following: the DSC and derivatives thereof, multiple DNA polymerases, multiple blocking molecules, and multiple concentrations of nucleic acid complex. The methods and compositions of the present invention can be adapted with other nucleic acid complexes and other polymerases to improve the performance of a nucleic acid amplification reaction.

According to the present invention, the nucleic acid complex can have a melting temperature up to about 90° C. The range of melting temperatures for the nucleic acid complex can be greater than about 25° C., for example from about 45° C. to about 75° C., or for example from about 45° C. to about 55° C. (e.g., a range between about 25° C. to about 90° C.). In the most preferred embodiment the nucleic acid complex has a melting temperature of about 48.9° C. This range of melting temperatures is useful in various amplification reactions known to those skilled in the art and as set forth herein.

Melting temperatures (Tₘ) for the nucleic acid complex are calculated using the nearest-neighbor thermodynamic parameters as provided by Integrated DNA Technologies, 1710 Commercial Park, Coralville, Iowa 52241 USA, using the calculations as described by Owczarzy, R. et al., Biochemistry, 2004 Mar. 30; 43(12):3537-54 and in Owczarzy, R. et al., Biochemistry, 2008 May 13; 47(19):5336-53 which are incorporated herein by reference.

The nucleic acid complex can have a range of concentrations for use in a variety of applications. The range of concentrations for the nucleic acid complex can be greater than about 2 μM DSC to 5,000 U/mL of polymerase, up to 2 mM DSC for every 5,000 U/mL of polymerase. More preferably the range is from about 20 μM DSC to 5,000 U/mL of polymerase, up to 200 μM DSC for every 5,000 U/mL of polymerase. In an embodiment, the most preferred concentration of DSC to polymerase is about 5,000 U/mL Taq to 200 μM DSC.

The specific activity of Taq DNA polymerase was measured using a 2-fold serial dilution method. Dilutions of enzyme were made in a reduced-glycerol (5%) containing Taq-B DNA Polymerase storage solution ([Taq-B]f=0.009-0.0001 μg/μL) and added to 50 μL reactions containing 12.5 μg Calf Thymus DNA, 25 mM TAPS (pH 9.3), 50 mM KCl, 1 mM DTT, 4 mCi/mL 3H-dTTP and 200 μM dNTPs. Reactions were incubated 10 minutes at 75° C., plunged on ice, and analyzed using the method of Sambrook and Russell (Molecular Cloning, v3, 2001, pp. A8.25-A8.26).

The present invention provides for a novel isolated nucleic acid complex, Table 1, derived, for example from a double stranded DNA oligonucleotide with a blocking molecule. The nucleic acid complex demonstrates the ability to inhibit the production of non-specific amplification products or the amplification of nontarget oligonucleotides due to side-reactions, such as mispriming of a background DNA and/or primer oligomerization. Amplification reactions containing the nucleic acid complex were found to enhance the production of the target oligonucleotide. Additionally the nucleic acid complexes of Table 1 have a 30% sequence identity with each other.

TABLE 1

| Seq ID No. | Name | Sequence (5′-3′) | Tₘ (° C.) |
|---|---|---|---|
| 1 | DSC1 | GCC AAT CCT ACG CC/InvT/ | 51.5 |
| 2 | DSC1-1 | GCC AAT CCT ACG CC/Phosph/ | 49.6 |
| 3 | DSC1-2 | GCC AAT CCT ACG CC/hexanediol/ | 49.6 |
| 4 | DSC2 | GCC GGC CAA TGT/InvT/ | 49.6 |
| 5 | DSC3 | CCT GAC AAT GCC GCG/InvT/ | 56.2 |
| 6 | DSC3-1 | CCT GAC AAT GCC GCG/hexanediol/ | 54.3 |
| 7 | DSC5 | AGC GGA TAA CAA TAT CAC A/InvT/ | 48.9 |
| 8 | DSC6 | GCC AAT CAT/InvT/ | 26.0 |
| 9 | DSC7 | GCC AAT CCT A/InvT/ | 30.7 |
| 10 | DSC8 | GCC AAT CCT AC/InvT/ | 36.8 |
| 11 | DSC9 | GCC AAT CCT ACG/InvT/ | 43.0 |
| 12 | DSC10 | GCC AAT CCT ACG C/InvT/ | 47.8 |
| 13 | DSC11 | GCC AAT CCT ACG CCT CC/InvT/ | 57.1 |
| 14 | DSC12 | GCC AAT CCT ACG CCT CCG T/InvT/ | 60.0 |
| 15 | DSC13 | GCC AAT CCT ACG CCT CCG TGA CGA TCC/InvT/ | 66.6 |
| 16 | DSC14 | GCC AAT CCT ACG CCT CCG TGA CGA TCC GCT C/InvT/ | 70.8 |

The sequences of preferred nucleic acid complexes encompassed by this invention in one embodiment are shown in Table 1. Abbreviations used in the table are: “InvT”=inverted deoxythymidine (dT); “Phospho”=phosphate group. The nucleic acid complexes show similarity to many sequences having an E-value below 1 as identified in a similarity search using BLAST (Altschul, S. F., et al., J. Mol. Biol., 215: 403-410 (1990)).

The terms “nucleic acid complex” and “Double Stranded Complex” (DSC), as used herein have the same meaning and are used interchangeably.

The invention further pertains to a nucleic acid complex wherein double stranded oligonucleotides about 16 nucleotides in length are made with a blocking molecule attached to either end, with a sequence for an optimal melting temperature, while having a high percentage of GC nucleotides at either end of the double stranded nucleic acid. The invention pertains to nucleic acid complexes which have been modified in one or more the following ways: to prevent their extension in an amplification reaction, to have a melting temperature that prevents the production of non-specific amplification products, to have a high percentage of GC at either end, which have a higher melting temperature than AT bonds and consequently are better able to maintain the nucleic acid complex as a double strand. The invention further pertains to storage buffer containing a double stranded nucleic acid with a blocking molecule, and a polymerase; and also to a reaction buffer comprising a nucleic acid complex.

The enhanced stability of the double stranded nucleic acid complex allows their use under conditions which would be prohibitive of other hot start methods, because the double stranded nucleic acid complex is not irreversibly denatured at elevated temperatures, thereby increasing the opportunities the nucleic acid complex can be employed to reduce non-specific amplification products. For example, amplification in a multiplex PCR with multiple specific primers, the opportunity for non-specific amplification products has a negative influence on the yield of the reaction, while chemically modified and antibody type hot start methods are mostly deactivated after the initial denaturing heat step, the nucleic acid complex of the present invention will continue to interact during the first several cycles of amplification when the reaction mixture is most vulnerable to non-specific priming. Additionally, the nucleic acid complex can be used, but is not limited to, isothermal amplification reactions, Variable Number Tandem Repeats (VNTR) PCR, asymmetric PCR, long PCR, nested PCR, quantitative PCR, touchdown PCR, assembly PCR, colony PCR, reverse transcription PCR, ligation-mediated PCR, and methylation-specific PCR.

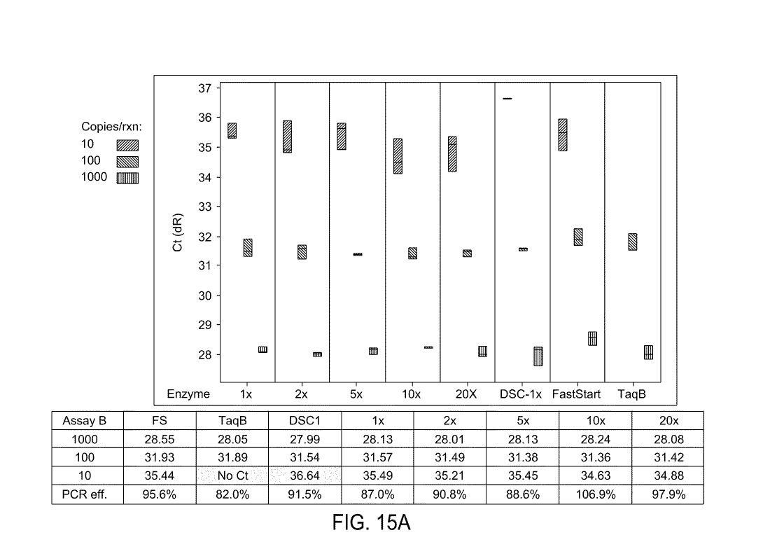

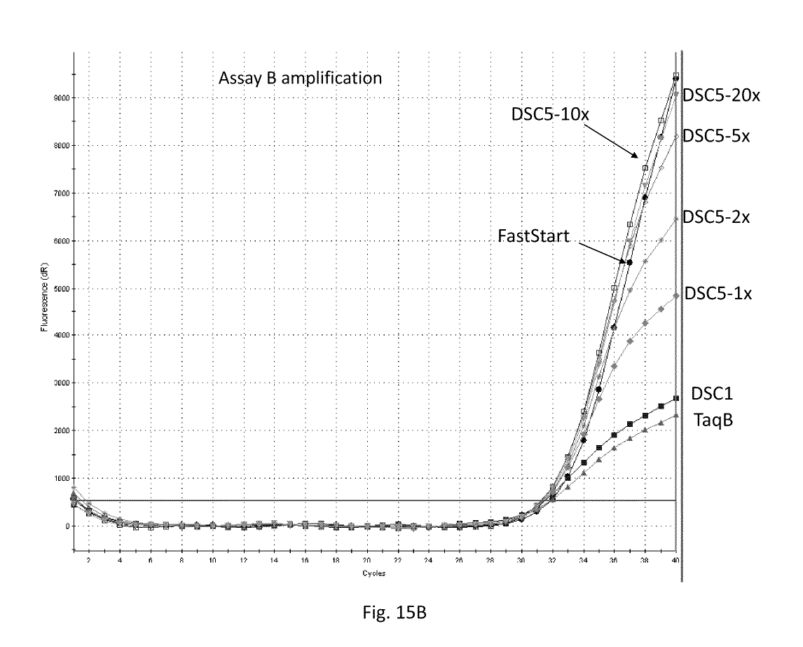

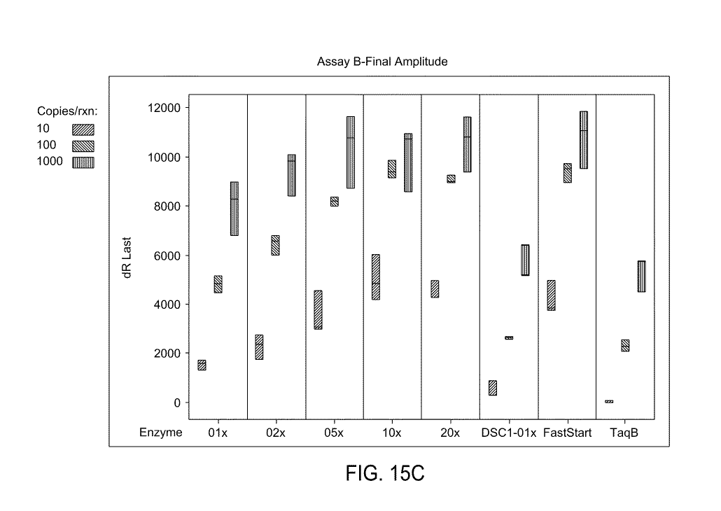

The use of a double stranded nucleic acid complex in hot-start PCR reactions surprisingly improves the amplification yield of the desired target sequences while also significantly reducing off-target amplification of unwanted sequences. This is accomplished by reducing polymerase activity at room temperature; both as compared to a typical hot-start PCR reaction not employing DSC technology as disclosed herein (see Example 5 and FIGS. 14-15). In one embodiment, use of DSC in a hot-start PCR reaction provides at least a two-fold (2×) improvement in yield. In another embodiment, such use provides at least a five-fold (5×) improvement in yield, while in another embodiment, such use provides a seven-fold (7×) or ten-fold (10×) or higher improvement in yield. In an embodiment, the increased amount in a yield ranges from about a 2× increase to about a 20× increase (e.g., a 2×, 3×, 4×, 5×, 6×, 7×, 8×, 9×, 10×, 15× or 20× increase), as compared to an amount of amplified target nucleic acid molecules that were not subjected to the methods or compositions of the present invention. Similarly, in one embodiment, use of DSC in a hot-start PCR reaction provides at least a fifty percent (50%) reduction in polymerase activity at room temperature (e.g., between about 20° C. and about 25° C.). In yet another embodiment, such use provides at least a seventy percent (70%) reduction in polymerase activity at room temperature, while in still another embodiment, such use provides an eighty percent (80%) or higher reduction in polymerase activity at room temperature. In an aspect, the present invention provides a reduction in polymerase activity at a temperature between about 20° C. and 25° C., wherein the reduction ranges between about 50% to about 90% (e.g., about a 50%, 60%, 70%, 80% or 90% reduction), as compared to polymerase activity in which the target nucleic acid molecules are not subjected to the DSC of the present invention. Methods for assessing polymerase activity are well known in the art, and include labeled-nucleotide incorporation assays. Briefly, a detectable label can be incorporated into the nucleic acid molecule and an assay can be performed to measure the activity of the polymerase e.g., on an automated fluorescence-based sequencing apparatus e.g., from Applied Biosystems (Life Technologies Corporation, Carlsbad, Calif.). Examples of detectable labels include fluorescent dyes, streptavidin conjugate, magnetic beads, dendrimers, radiolabels, enzymes, colorimetric labels, digoxigenin, biotin, nanoparticles, and/or nanocrystals). Methods for incorporating labels are known in the art. Several assays for measuring polymerase assays exist. One example includes, as mentioned above, labeled-nucleotide incorporation assays, in which a DNA polymerase assay takes advantage of the ability of DNA dependent DNA polymerases to incorporate modified nucleotides into freshly synthesized DNA. Certain assays can be radioactive while others use non-radioactive labels. (e.g., Cat. No. 1 669 885 Roche Molecular Biochemicals, Indianapolis, Ind.). The labeled nucleotides in an optimized ratio are incorporated into the same DNA molecule by the DNA polymerase activity. The detection and quantification of the synthesized DNA as a parameter for DNA polymerase activity can be assessed using any number of detection methods.

Similarly, methods for assessing yield of PCR reactions are well known in the art, and include quantitative-PCR (qPCR) methods. For example, the yield can also be labeled and after each cycle of PCR, a real-time PCR instrument can measure levels of the label (e.g., fluorescence). Amounts of yield can be determined by comparing the results to a standard curve produced by real-time PCR of serial dilutions (e.g. undiluted, 1:4, 1:16, 1:64) of a known amounts. Nolan, Tania et al., Nature Protocols 1:1559-1582 (2006).

The term “primer” refers to an oligonucleotide capable of acting as a point of initiation of DNA synthesis under conditions in which synthesis of a primer extension product complementary to a nucleic acid strand in induced.

The nucleic acid complex of the invention can be DNA or RNA, including double stranded RNA or DNA. In another embodiment of the invention the oligonucleotides of the nucleic acid complex can be composed of modified nucleotides or synthetic nucleic acid molecules. Modifications include, but are not limited to, those which provide other chemical groups that incorporate additional charge, polarizability, hydrogen bonding, electrostatic interaction, and fluxionality to the nucleic acid or to the nucleic acid complex as a whole. Modifications include, but are not limited backbone modifications, methylations, 3′ and 5′ modifications. The blocking molecule, in an aspect, comprises one or more nucleotide analogs with modified bases, modified sugars and or modified phosphate groups.

In another embodiment, the nucleic acids of the complex are for example, but not limited to, a modified nucleic acid with an abasic moiety, an inverted abasic moiety, an inverted nucleotide moiety, an inverted deoxynucleotide 3′ to 3′ linkage, a disaccharide nucleotide, locked nucleic acids, 2′-Amino pyrimidines, 2′-Fluoro pyrimidines, 2′-O-methyl nucleotides, Boranophosphate internucleotide linkages, 5-Modified pyrimidines, 4′-Thio pyrimidines, Phosphorothioate internucleotide linkages.

In another embodiment the nucleotides of the nucleic acid complex are synthetic oligonucleotides. Synthetic oligonucleotides have widespread use in various fields such as in molecular biology, including genetic engineering; in therapeutics, for example for antisense oligonucleotides; for diagnostics and to make catalysts as ribozymes. PCR technology, for example, routinely employs oligonucleotides as primers for amplification of genetic material and synthetic genes are made for various purposes including optimization of codon usage for efficient expression. Useful synthetic oligonucleotides include polymers containing natural ribonucleotides and deoxynucleotides as well as polymers containing modified nucleotides such as base-modified, sugar-modified and phosphate-group modified nucleotides.

Preferably, the nucleic acid complexes are comprised of a double stranded oligonucleotide about 9 to 40 nucleotides in length, and even more preferably about 14 to 20 nucleotides in length. In the most preferred embodiment, the oligonucleotides of the invention are 16 to 19 nucleotides in length.

DNA polymerases isolated from species of archaebacteria (e.g. PFU DNA polymerase) often stall (in activity) when they encounter a uracil (U) base in the nucleotide sequence they are reading. See, e.g. Hogrefe et. al. Proc. Natl. Acad. Sci. USA, 99: 596-602 (2002); Fogg et al., Nat. Struct. Biol., 9(12): 922-927 (2002). Thus, in accordance with the present invention, the introduction of one or more uracil (U) bases into a double stranded nucleic acid complex disclosed herein significantly reduces undesirable polymerase activity at room temperature (prior to start of PCR reactions) when a DNA polymerase from a species of archaebacteria is employed. Accordingly, in one embodiment of the invention, there is provided a double stranded nucleic acid complex as disclosed herein but further comprising at least one uracil (U) base. In another embodiment, the method disclosed herein employs a DSC comprising such uracil base and at least one thermostable DNA polymerase derived from a species of archaebacteria. In one embodiment, such use of a uracil-containing DSC in a hot-start PCR reaction provides at least a fifty percent (50%) reduction in polymerase activity at room temperature. In yet another embodiment, such use provides at least a seventy percent (70%) reduction in polymerase activity at room temperature; while in still another embodiment, such use provides an eighty percent (80%) or higher reduction in polymerase activity at room temperature. In an aspect, the present invention use of a uracil-containing DSC provides a reduction in polymerase activity at a temperature between about 20° C. and 25° C., wherein the range is between about 50% to about 90% (e.g., about 50%, 60%, 70%, 80% or 90% reduction), as compared to polymerase activity in which the target nucleic acid molecules are not subjected to the DSC of the present invention. Methods for assessing polymerase activity are well known in the art, and include labeled-nucleotide incorporation assays.

The DSC of the present invention is believed to have at least two methods of inhibition. First, the DSC effectively acts as an inhibitor of the DNA polymerase on its own, without the Inverted dT. In solution with the DNA polymerase, the DNA polymerase will naturally bind to the DSC of the present invention, though some double stranded DNA sequences seem to offer more effective inhibition than others, see Kainz et al. BioTechniques 28:278-282 (February 2000). Second, when the PCR reaction begins its first cycle, the DSC can be dislodged from the DNA polymerase when each strand separates, because the temperature of the reaction exceeds the melting temperature of the DSC. On cooling, each strand of the DSC will typically reform to hybridize with its complement where it inhibits the polymerization activity of the DNA polymerase. However, if the DSC by chance does hybridize to the target template DNA, the presence of the inverted dT will effectively inhibit the DNA polymerase from extending and forming a competing secondary product because the inverted dT lacks a 3′ hydroxyl group that is necessary for the DNA polymerase to add additional nucleotides. Furthermore, the inverted dT is protected from exonucleases. Owing to its unusual structure, an exonuclease cannot remove the inverted dT, the degradation of which would otherwise allow the DSC to non-specifically anneal and act as a primer for DNA extension.

In one embodiment, the nucleic acid molecules of the invention are “isolated”; as used herein, an “isolated” nucleic acid molecule or nucleotide sequence is intended to mean a nucleic acid molecule or nucleotide sequence which is not flanked by nucleotide sequences which normally (in nature) flank the gene or nucleotide sequence (as in genomic sequences) and/or has been completely or partially purified from other transcribed sequences. For example, an isolated nucleic acid of the invention can be substantially isolated with respect to the complex cellular milieu in which it naturally occurs. In some instances, the isolated material will form part of a composition, buffer system or reagent mix. Thus, an isolated nucleic acid molecule or nucleotide sequence of the nucleic acid complex can include a nucleic acid molecule or nucleotide sequence which is synthesized chemically or by recombinant means. Also, isolated nucleotide sequences include partially or substantially purified nucleic acid molecules in solution.

The present invention, in one embodiment, includes an isolated nucleic acid molecule having a nucleic acid sequence of any one of SEQ ID NOs:1-16 comprising a covalently attached blocking molecule; a nucleic acid sequence having between about 80% and about 100% of contiguous nucleotides of any one of SEQ ID NO: 1-16; a nucleic acid sequence having between about 7 and about 20 contiguous nucleotides of any one of SEQ ID NO: 1-16; a complement thereof; and any combination thereof.

As used herein, the terms “DNA molecule” or “nucleic acid molecule” include both sense and anti-sense strands, cDNA, complementary DNA, recombinant DNA, RNA, wholly or partially synthesized nucleic acid molecules, PNA and other synthetic DNA homologs. A nucleotide “variant” or “derivative” is a sequence that differs from the recited nucleotide sequence in having one or more nucleotide deletions, substitutions or additions so long as the molecules block non-specific amplification during PCR.

Also encompassed by the present invention are nucleic acid sequences, DNA or RNA, PNA or other DNA analogues, which are substantially complementary to the DNA sequences. As defined herein, substantially complementary, analog or derivative means that the nucleic acid need not reflect the exact sequence of the sequences of the present invention, but must be sufficiently similar in sequence to permit hybridization with nucleic acid sequence of the present invention under high stringency conditions. For example, non-complementary bases can be interspersed in a nucleotide sequence, or the sequences can be longer or shorter than the nucleic acid sequence of the present invention, provided that the sequence has a sufficient number of bases to reduce non-specific amplification during PCR.

In another embodiment, the present invention includes molecules that contain at least about 7 to about 20 contiguous nucleotides or longer in length (e.g., 7, 8, 9, 10, 11, 12, 13, 14, 15, 16, 17, 18, 19, or 20) of any nucleic acid molecules described herein, and preferably of SEQ ID NO: 1-16. Alternatively, molecules of the present invention includes nucleic acid sequences having contiguous nucleotides of about 60% and about 100% of the length of any one of the sequences described herein, and preferably of SEQ ID NO: 1-16.

The invention also pertains to nucleic acid complexes which have a substantial identity with the sequences of the nucleic acid complexes described herein; particularly preferred are nucleotide sequences which have at least about 10%, preferably at least about 20%, more preferably at least about 30%, more preferably at least about 40%, even more preferably at least about 50%, yet more preferably at least about 70%, still more preferably at least about 80%, and even more preferably at least about 90% identity, and still more preferably 95% identity, with nucleotide sequences described herein. Particularly preferred in this instance are nucleic acid complexes having an activity of a nucleic acid complex as described herein.

To determine the percent identity of two nucleic acid complexes, the sequences are aligned for optimal comparison purposes (e.g., gaps can be introduced in the sequence of a first nucleotide sequence). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences (i.e., % identity=number of identical positions/total number of positions×100).

The nucleic acid complexes described herein (e.g., a nucleic acid complex as shown in Table 1) are useful in reducing non-specific amplification in an amplification reaction, e.g. PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202.

The term, “amplifying,” refers to increasing the number of copies of a specific or target polynucleotide. For example, PCR is a method for amplifying a polynucleotide sequence using a polymerase and two oligonucleotide primers, one complementary to one of two polynucleotide strands at one end of the sequence to be amplified and the other complementary to the other of two polynucleotide strands at the other end. Because the newly synthesized DNA strands can subsequently serve as additional templates for the same primer sequences, successive rounds of primer annealing, strand elongation, and dissociation produce rapid and highly specific amplification of the desired sequence. PCR also can be used to detect the existence of the defined sequence in a DNA sample. The DNA of the sample is amplified or replicated, in one embodiment, with PCR. Methods of PCR are known in the art and are described for example in Mullis, K. B. Scientific American 256:56-65 (1990).

Briefly, PCR is performed with the use of a DNA polymerase enzyme and include, for example, one that is isolated from a genetically engineered bacterium. Preferred polymerase enzymes are derived from thermostable organisms, such as Thermus aquaticus (Taq). Additional polymerases are described herein, and encompass thermostable archeabacterial polymerases. The polymerase, along with the primers, the DSC complex of the present invention, and a supply of the four nucleotide bases (adenine, guanine, cytosine and thymine) are provided. Under certain conditions (e.g., 95° C. for 30 seconds), the DNA is denatured to allow the strands to separate. As the DNA solution cools, the primers bind to the DNA strands, and then the solution is heated to promote the Taq polymerase to take effect. Mullis, K. B. Scientific American 256:56-65 (1990).

Other suitable amplification methods include the ligase chain reaction (LCR) (see Wu and Wallace, Genomics, 4:560 (1989), Landegren, et al., Science, 241:1077 (1988), transcription amplification (Kwoh, et al., Proc. Natl. Acad. Sci. USA 86:1173 (1989)), and self-sustained sequence replication (Guatelli, et al., Proc. Nat. Acad. Sci. USA, 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be radiolabelled and used as a probe for screening a library or other suitable vector to identify homologous nucleotide sequences. Corresponding clones can be isolated, DNA can be obtained following in vivo excision, and the cloned insert can be sequenced in either or both orientations by art recognized methods, to identify the correct reading frame encoding a protein of the appropriate molecular weight. For example, the direct analysis of the nucleotide sequence of homologous nucleic add molecules of the present invention can be accomplished using either the dideoxy chain termination method or the Maxam Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)). Using these or similar methods, the protein(s) and the DNA encoding the protein can be isolated, sequenced and further characterized.

The nucleic acid complexes of the invention can also be used for amplification of RNA, such as methods for amplification of mRNA including synthesis of the corresponding cDNA.

In a further embodiment the DNA polymerase is chosen from Taq DNA polymerase, BST DNA Polymerase, PFU DNA polymerase, Klenow DNA polymerase, T7 DNA polymerase, T4 DNA polymerase, Phi29 DNA polymerase, RB69 DNA polymerase. Thermostable DNA polymerases derived from species of archaebacteria are commercially available (e.g. New England Biolabs, Inc.; Stratagene, Inc.), and include 9∘N DNA polymerase and Vent DNA polymerase.

In one embodiment, the nucleic acid complex binds to a thermostable DNA polymerase. In a second embodiment the nucleic acid complex only momentarily binds with a DNA polymerase, the DNA polymerase rapidly becomes unbound and rebinds either to the same nucleic acid complex or another nucleic acid complex. In another embodiment, the nucleic acid complex reduces the amount of non-specific amplification product in an amplification reaction at a temperature below the melting temperature of the nucleic acid. In another embodiment the nucleic acid complex is a DNA construct which inhibits the activity of a thermostable DNA polymerase in an amplification reaction, wherein the DNA construct has a melting temperature of approximately 51.5° C., or the DNA construct reduces the amount of non-specific amplification product.

Additionally, the nucleic acids of the nucleic acid complex comprise a blocking molecule. In a preferred embodiment, the blocking molecule prevents the extension of the nucleic acid complex by a polymerase. In an alternative embodiment, the blocking molecule prevents extension by a particular polymerase, such as a DNA polymerase. The blocking molecule can be attached to either the 5′ or 3′ end of the nucleic acid. In another preferred embodiment, the blocking molecule provides resistance to 5′ and/or 3′ exonuclease digestion. In the most preferred embodiment, the blocking molecule is an inverted deoxythymidine covalently attached to the 3′ terminus, and prevents extensions by a thermostable DNA polymerase and provides resistance to 3′ exonuclease activity.

In an embodiment, the methods and reagents use double stranded oligonucleotides blocked at the 3′ hydroxyl terminus. In the preferred embodiment, Taq DNA polymerase is combined with a double stranded oligonucleotide that is capped with a blocking molecule. The blocking molecule is covalently attached to the oligonucleotide. The blocking molecules are not able to be removed by incubation in the amplification reaction at an elevated temperature. The combination of the double stranded oligonucleotide and the blocking molecule is referred to herein as Double Stranded Complex (DSC). Because of the blocking molecule, in one embodiment, the DSC will not be degraded by any contaminating 3′ exonuclease nor can the nucleic acid be extended by the polymerase. If by chance the single strands of the DSC happen to hybridize to the reaction primers or the template, the blocking molecule prevents the DSC from acting as an unintended primer and forming a competing, contaminating product. Accordingly, the present invention provides a means for improving the performance of a nucleic acid amplification reaction. This invention pertains, but is not limited, to nucleic acid complexes composed of double stranded oligonucleotides that bind to a DNA polymerase and prevent the production of non-specific amplification products. Each strand of the double stranded nucleic acid complex comprises a blocking molecule, which protects the nucleic acid complex from exonuclease degradation and also prevents the nucleic acid complex from itself becoming the source of unspecific oligonucleotide priming.

Blocking molecules are defined as to include any molecule which prevents extension of the nucleic acid complex by a polymerase. Blocking molecules can also be resistant to exonuclease degradation. In a further preferred embodiment, the blocking molecule prevents extension by a polymerase as well as prevents excision by an exonuclease. The blocking molecule can be placed at either the 3′ or 5′ terminus of the nucleic acid complex. In the most preferred embodiment the blocking molecule is an Inverted dT. Inverted dTs are synthetic nucleotides of deoxythymidine whose bonds between the ribose structure and the thymidine base are in a position that is inverted from the standard deoxythymidine:

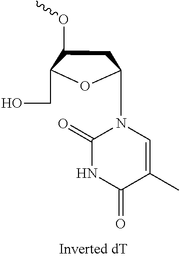

Examples of a blocking molecule include, but are not limited to, deoxythymidine, dideoxynucleotides, 3′ phosphorylation, hexanediol, spacer molecules, 1′2′-dideoxyribose, 2′-0-Methyl RNA, Locked Nucleic Acids (LNAs), and synthetic or natural molecules which prevent extension of the nucleic acid complex and/or are resistant to excision by an exonuclease.

The present invention also relates to a DNA construct comprising an isolated nucleic acid molecule of the present invention, in solution with a storage buffer. Such storage buffer, for example, includes a TRIS buffer, MOPS, or HEPES buffer. Further, the present invention relates to a storage buffer comprising the nucleic acid complex of the invention and a DNA polymerase.

Accordingly, the nucleic acid molecule of the present invention, in one embodiment, is a double stranded nucleic acid construct with a blocking molecule attached selected from the group consisting of:
a) a nucleic acid composed of deoxyribonucleic acid;
b) a nucleic acid with a blocking molecule in the middle of the nucleic acid;
c) a nucleic acid with a blocking molecule attached to either end of the nucleic acid;
d) a nucleic acid with a blocking molecule attached to the 3′ end of the nucleic acid;
e) a nucleic acid which reduces non-specific amplification;
f) a nucleic acid with the nucleic acid sequence of any of the sequences from Table 1;
g) a nucleic acid with a melting temperature of approximately 48.9° C. to 51.5° C.;
h) a nucleic acid with a GC content of approximately 64.3%;
i) a nucleic acid wherein the nucleic acid has one or more modified nucleotides;
j) a nucleic acid wherein the one or more artificial nucleic acids are contained therein,
k) a nucleic acid wherein the blocking molecule is spacer molecule;
m) a nucleic acid wherein the blocking molecule is an inverted nucleotide;
n) a fragment or derivative of a), b), c), d), e), f), g), h), i), j), k), l), m).

In another embodiment, the present invention relates to an isolated nucleic acid by itself, and in various compositions, such as:
a) nucleic acid comprising the sequence of DSC1;
b) a storage buffer containing the nucleic acid of DSC1;
c) a reaction buffer containing the nucleic acid of DSC1; or
d) a DNA construct having at least 90% sequence identity with the nucleic acid sequence of DSC1; and e) a fragment or derivative of a), b) or c).

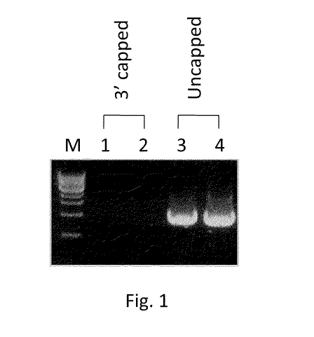

Example 1 (FIG. 1) describes a PCR amplification which demonstrates that the DSC of the invention effectively prevents the extension by a DNA polymerase. The inhibitory molecules incorporated at the 3′ end of the oligonucleotides cap the free hydroxyl end resulting in no product amplification.

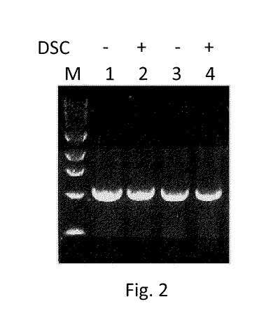

Example 1 (FIG. 2) also describes a PCR amplification which demonstrates that the DSC of the invention does not inhibit the successful amplification of a 1.1 kb region of the pUC19 plasmid. The presence of the double stranded molecule with capped 3′ ends having the same sequence as the forward and reverse reaction primers does not inhibit the outcome of the reaction.

Example 2 describes a number of PCR amplifications, including standard PCR, commercially available hot start PCR, and PCR in the presence of the double stranded complex of the invention without manual hot start conditions. Example 2 describes the detection of 1.9 kb region of Lambda Phage DNA in the presence of E. coli genomic DNA as a competing foreign DNA.

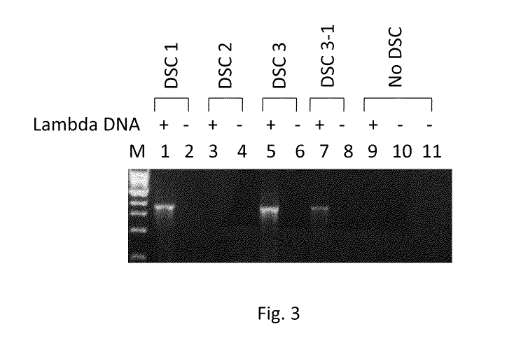

FIG. 3 illustrates the results of the PCR amplification in the presence of DSC1, DSC2, DSC 3, DSC3-1 molecules. PCR amplifications were carried out with 10,000 copies of lambda phage DNA, in odd number lanes. All reactions contain 1 ng of E. coli genomic DNA as a competing foreign DNA. Reactions in which there was no DSC molecule added, no amplification of product was achieved. When the DSC molecule was added in lanes 1, 5, 7, amplification of product was achieved and yield was not comprised by non specific amplification. The presence of the DSC molecule facilitates the detection of the target band without compromising the yield.

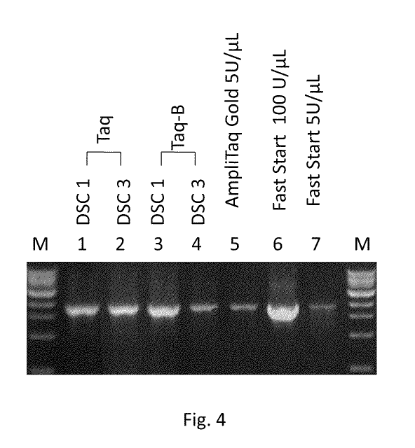

FIG. 4 depicts the comparison in amplification of a 1.9 kb region from Lambda DNA. All PCR reactions were performed in the presence of Lambda DNA and 1 ng of contaminating E. coli genomic DNA. Lanes 1-4 illustrate the amplification of product using Taq and Taq-B DNA polymerase in the presence of DSC molecules. Taq-B has stabilizers in its storage buffer. In lanes 5-7 amplification was carried out with commercially available, chemically modified hot start DNA polymerases. The yield of amplified product in the presence of the DSC molecule is comparable or greater than the yield obtained with the chemically modified Taq.

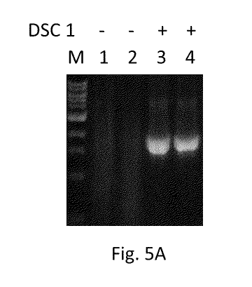

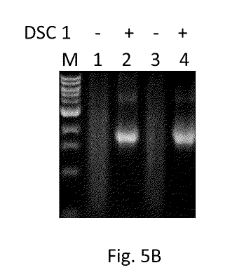

FIGS. 5A and B emphasize the amplification of a 1.9 kb region of Lambda DNA in the presence of DSC molecule. In FIG. 5A the PCR amplification was carried out with forward 5′CTGGCTGACATTTTCG-3′ (SEQ ID NO: 17) and reverse 5′TATCGACATTTCTGCACC-3′ (SEQ ID NO: 18) primers that have a lower melting Temperature than the primers used in FIGS. 3 and 4. In FIG. 5B the amplification was carried out with forward 5′GAAGTCAACAAAAAGGAGCTGGCTGACATTTTCG-3′ (SEQ ID NO: 19) and reverse 5′CAGCAGATACGGGATATCGACATTTCTGCACC-3′ (SEQ ID NO: 20) primers that have a higher melting Temperature than the primers used in FIGS. 3 and 4. PCR amplification was performed with 0.523 pg of Lambda DNA and 1 ng of E. coli genomic DNA. Reaction condition were initial denaturation at 95° C. for 5 min, followed by 40 cycles of 95° C. for 40 s, of either 48° C. or 61° C. for 30 s, 72° C. for 2 min, final extension at 72° C. for 7 min. The results show that amplification of product was achieved in the presence of the DSC1 molecule. The melting Temperature of the reaction primers and annealing at lower/higher Temperature did not have an effect on the performance of Taq-B DSC.

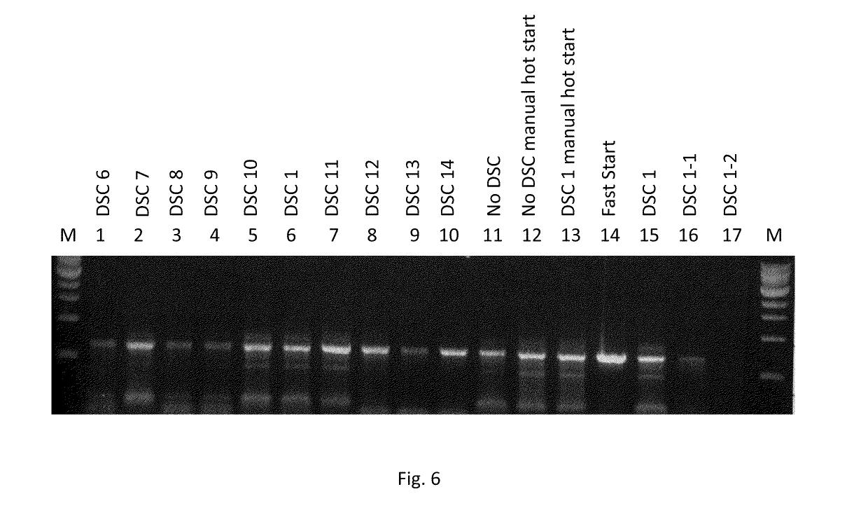

Example 3 describes the amplification of a 653-bp fragment of the β-actin gene of human placental DNA (FIG. 6) in standard PCR, manual hot start PCR, hot start PCR, and PCR in the presence of the DSC molecules. Under normal PCR, the yield of the desired band is compromised by the amplification of non specific bands (FIG. 6, lane 11).

Under conditions of manual hot start, the yield is increased but not the PCR specificity (FIG. 6, lane 12 and 13). The chemically modified polymerase is specific and it amplifies a robust band (FIG. 6, lane 14). In lanes 1-10, the melting temperatures of the DSC molecules are increasing by about 5° C. in each consecutive lane. Amplification performed in the presence of DSC molecules with low melting temperatures turned out low yield of the desired band (FIG. 6, lanes 1-4). The results of amplification carried out in the presence of the DSC molecule of mid melting Temperatures are similar to the ones obtained with amplification under manual hot start (FIG. 6, lanes 5-7). The addition of DSC molecules of high melting temperatures increased the specificity of the reaction and produced a robust desired band (FIG. 6, lanes 8-10). The difference in blocking molecule also affected the amplification of the desired band. The DSC molecules used in FIG. 6, lanes 16 and 17 were capped with a different blocking molecule at their 3′ end possibly interfering with the successful amplification of the reaction.

Example 4 describes a series of PCR reactions that amplify a 100-120 bp fragment of DNA A, B, and C at 1000 copies to 5 copies. PCR amplifications were performed under standard PCR, hot start PCR with chemically modified enzyme, and under non-hot start conditions in the presence of the DSC molecules of the invention. PCR amplification reactions run immediately after set-up were compared to those run after a 24 hour incubation period at ambient temperature.

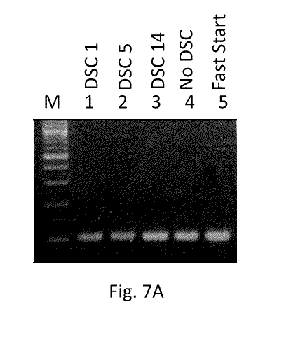

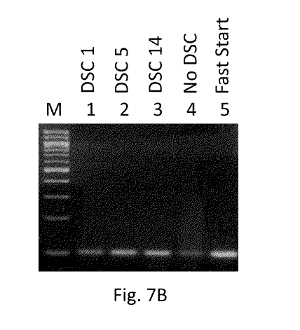

FIG. 7A depicts the amplification of a 100-bp product from DNA B using oligonucleotide mix B. All PCR amplifications contain 1000 copies of DNA B. The PCR amplification in FIG. 7A was performed immediately after set-up with no pre-incubation at bench top. The amount of the DNA present in the reactions is sufficient for the polymerase to amplify specifically without compromising the yield (FIG. 7A, lanes 1-5). There is single band present in all lanes that corresponds to the amplification of the desired product. Under the set-up in FIG. 7A, there is no need for the PCR amplification to be performed under hot start conditions.

In FIG. 7B the PCR amplification was performed after 24 hour incubation at ambient temperature of 23° C. Under normal PCR conditions, the amplification of the desired band is greatly reduced compared to the same amplification in lane 4 in FIG. 7A. The outcome is clearly different when the DSC molecules of the invention are added to the reaction. The presence of the DSC molecules in the reaction improves the amplification yield (FIG. 7B, lanes 1-3).

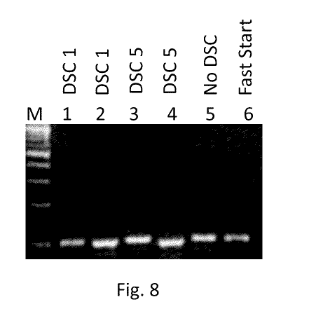

FIG. 8 illustrates the amplification of a 100-bp product from DNA B. All PCR amplifications were carried out with 5 copies of DNA. The PCR amplification was performed immediately after set-up with no pre-incubation at bench top. In lanes 1 and 3, the final concentration of the DSC molecule is 0.4 μM whereas in lanes 2 and 4, the final concentration if the DSC is 4 μM. The higher concentration of the DSC does not inhibit the outcome of the amplification. Reactions containing both concentrations of the DSC amplify a good amount of product.

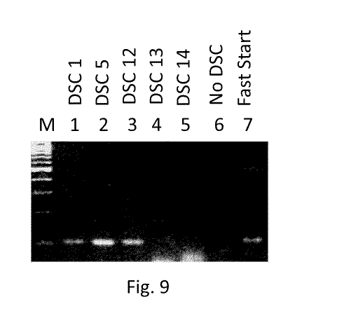

FIG. 9 illustrates the amplification of a 100-bp product from DNA B. All PCR amplifications were performed with 5 copies of DNA B. The PCR amplification was performed after 24 hour bench top incubation at ambient temperature. Under standard PCR conditions, the incubation period prevented the successful amplification of the desired product (FIG. 9, lane 6). Lanes 1-5 show the outcome of PCR amplifications in the presence of different DSC molecules at 4 μM final concentration. The amplification yield in the presence of DSC1 (FIG. 9, lane 1) is comparable to the yield obtained with the chemically modified enzyme (FIG. 9, lane 7). The yield obtained with DSC 5 and DSC12 (FIG. 9, lanes 2 and 3) is greater than yield obtained with the chemically modified enzyme (FIG. 9, lane 7). The outcome of amplification performed in the presence of DSC13 and DSC14 is similar to that obtained by PCR under standard conditions.

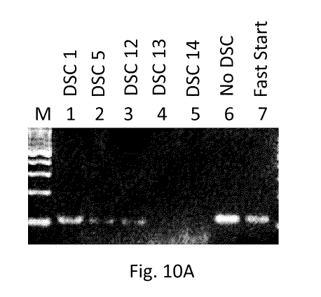

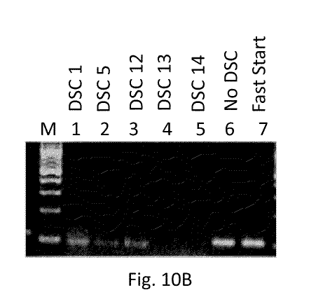

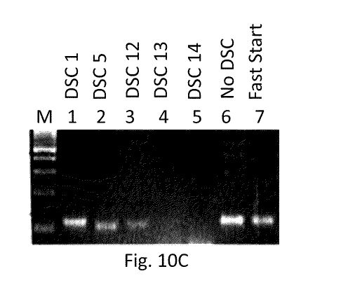

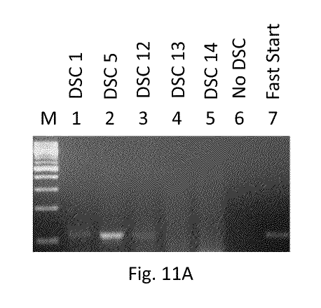

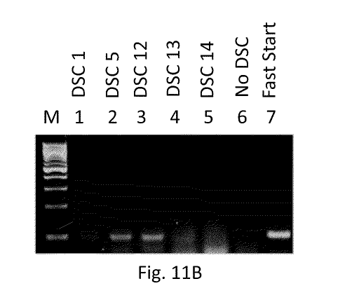

FIG. 10 A-C illustrate the amplification of a 100-120-bp product from DNA A-C respectively. All PCR amplifications were carried out with 5 copies of DNA. The PCR amplification was performed immediately after set-up with no pre-incubation at bench top. The final concentration of the DSC molecule is 4 μM (FIG. 10A-C, lanes 1-5). The higher concentration of the DSC does not inhibit the outcome of the amplification (FIG. 10A-C, lanes 1-3). In those reactions, there is a single band that corresponds to the amplification of the desired product. The outcome of the PCR performed in the presence of DSC13 and DSC14 did not result in the amplification of product, presumably because of their length. Under standard PCR conditions, there is a single band corresponding to the amplification of the desired product (FIG. 10A-C, lanes 6). Since the PCR amplification was performed immediately after set-up there was no need for the PCR to be performed under hot start conditions (FIG. 10A-C).

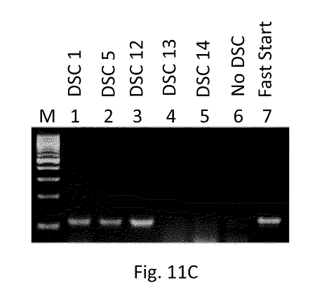

FIG. 11A-C depict the amplification of a 100-120-bp product from DNA A-C respectively. All PCR amplifications were carried out with 5 copies of DNA. The PCR amplification was performed after 24 hour bench top incubation at 23° C. The final concentration of the DSC molecule is 4 μM (FIG. 11A-C, lanes 1-5). The results of the PCR amplification performed in the absence of the DSC molecules were no amplification of product (FIG. 11A-C, lane 6). The outcome of amplification performed in the presence of DSC13 and DSC14 is similar to that obtained by PCR under non-hot start conditions (FIG. 11A-C, lanes 5 and 6).

In FIG. 11A, the amplification yield in the presence of DSC1 and DSC12 is similar to that obtained with the chemically modified polymerase (FIG. 11A, lanes 1, 3 and 7). There is enhanced yield obtained in the presence of DSC 5 (FIG. 11A, lane 2).

In FIG. 11B, amplification in the presence of the DSC1 results in no product formation (FIG. 11B, lane 1). However, amplification in the presence of DSC5 and DSC12 results in the detection of a single band corresponding to the target product (FIG. 11B, lanes 2 and 3).

In FIG. 11C, amplification in the presence of the DSC1, DSC5, and DSC12 result in product formation of comparable yield to that obtained with the chemically modified polymerase (FIG. 11C, lanes 1-3). However, amplification in the presence of DSC5 and DSC12 results in the detection of a single band corresponding to the target product.

The amplification in the presence of DSC 5 of the invention in assays illustrated in FIGS. 10 and 11 gives the best overall results.

Example 5 describes a series of qPCR reactions that amplify a 100-120-bp fragment of DNA A, B, C, and human placental DNA at 1280 copies to 5 copies. PCR amplifications were performed under standard PCR, hot start PCR with chemically modified enzyme, and under non-hot start conditions in the presence of the DSC molecules of the invention.

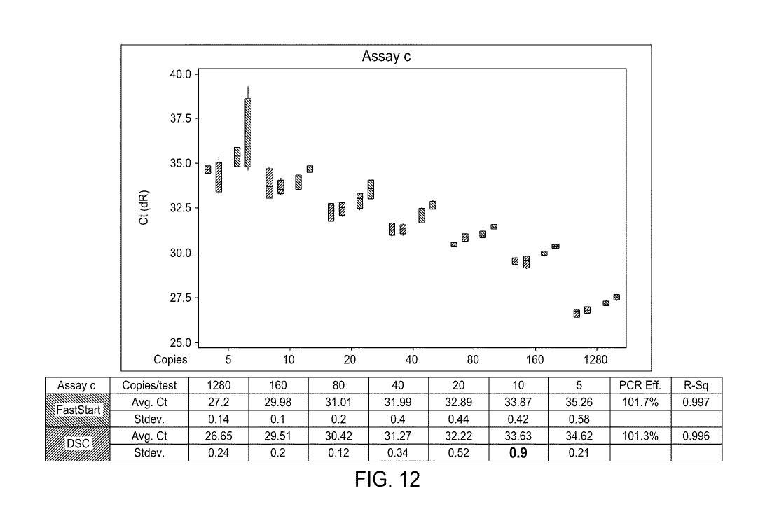

FIG. 12 depicts the real-time PCR analysis of the formation of 120-bp product from DNA C using detection by CY5 fluorescent dye. A side by side comparison was performed evaluating Taq-B DSC1 against a commercially available chemically modified Taq. The final concentration of DSC in each reaction is 0.4 μM. qPCR amplifications, which contained from 1280-5 copies of DNA C were performed in quadruplicate. Average Ct values for Taq DSC1 are lower than the ones obtained with the chemically modified Taq, at each copy level. Standard deviations are slightly higher for Taq DSC1 with at highest at 10 copy levels. The overall PCR efficiency and with R-squared values are comparable for both Taq DSC1 and the chemically modified Taq.

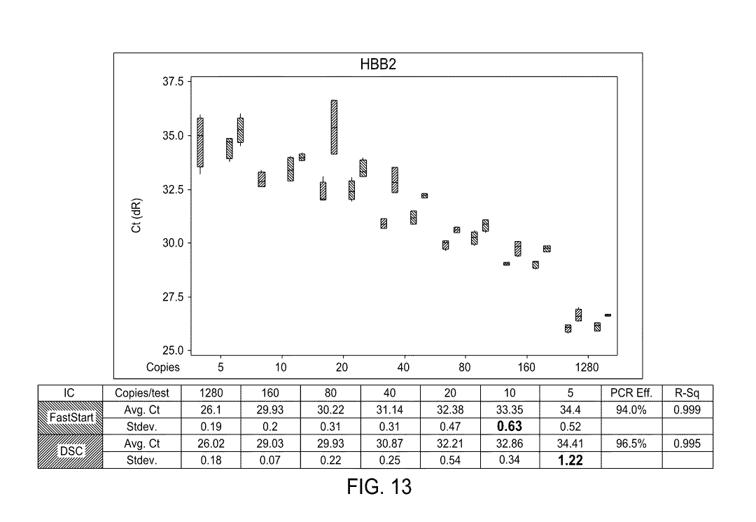

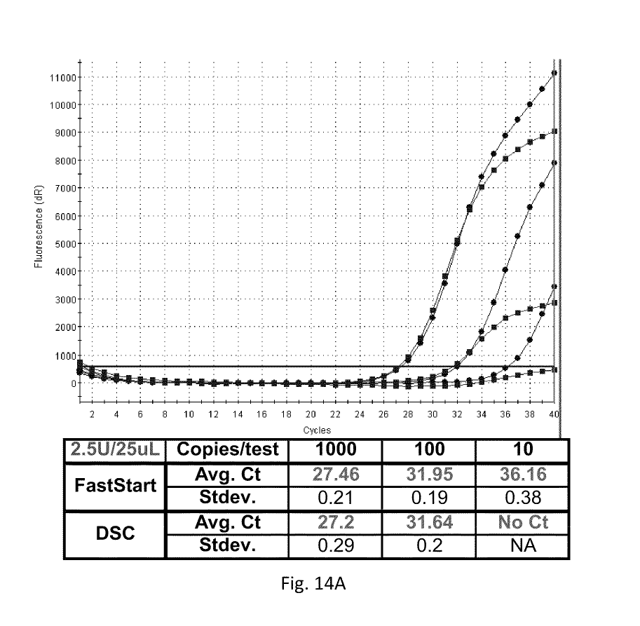

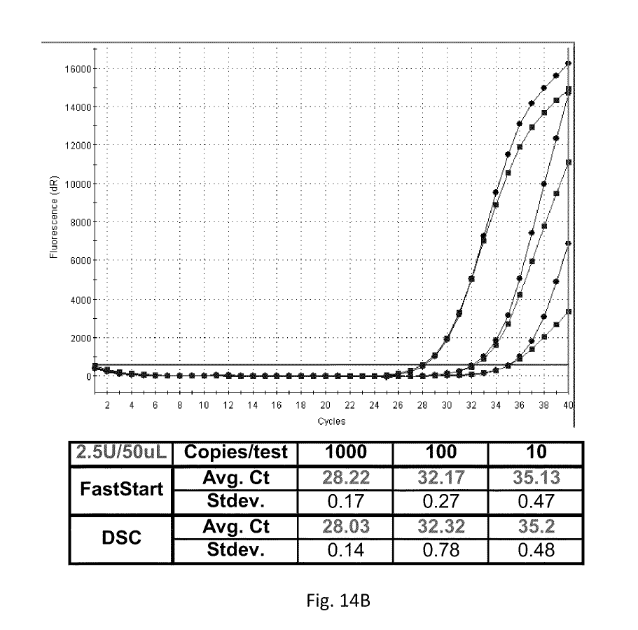

FIG. 13 depicts the real-time PCR analysis of the formation of 100-bp product of HBB2 from human placental DNA. A side by side comparison was performed evaluating Taq-B DSC1 against a commercially available chemically modified Taq. The final concentration of DSC1 in each reaction is 0.4 μM. qPCR amplifications, which contained from 1280-5 copies of human placental DNA, were performed in quadruplicate. Average Ct values for Taq-B DSC1 are lower than the ones obtained with the chemically modified Taq, at each copy level. Standard deviations are slightly lower too for Taq-B DSC1. At 10 copies, the chemically modified Taq has a standard deviation that is higher than the acceptable value of 0.6 whereas for Taq-B DSC1 that occurs at 5 copies. The overall PCR efficiency and with R-squared values are comparable for both Taq-B DSC1 and the chemically modified Taq.

FIGS. 14 A and B illustrate the real-time PCR analysis of the formation of 100-bp product from DNA B using detection by HEX fluorescent dye. FIG. 14A represents the amplification of product in 25 μL reaction with 2.5 U of Taq-B and 0.4 μM final concentration of DSC1. FIG. 14B shows the amplification curves of product in 50 μL reaction with 2.5 U of Taq-B and 0.2 μM final concentration of DSC1. Amplification reactions, which contained 1000, 100, and 10 copies of DNA B were performed in quadruplicate. By increasing the volume of the reaction, the final amplitudes at each copy level increased for both Taq-B DSC1 and the chemically modified Taq suggesting that using less units/mL of enzyme should be used. The increase is more dramatic for Taq-B DSC1 at the 10 copy level. In those reactions (FIG. 14A), there is no detectable Ct value (amplification below the threshold value) compared to a measurable value in FIG. 14B.

FIG. 15A-C illustrate the real-time PCR analysis of the formation of 100-bp product from DNA B using detection by HEX fluorescent dye. Reactions, which contained 1000, 100, and 10 copies of DNA B were performed in quadruplicate. In this assay, the qPCR amplifications performed using Taq-B, Taq-B DSC1, Taq-B DSC5, and chemically modified Taq are compared. The final concentration of DSC5 molecule in reaction was noted from 1×-20×, where 1× was 0.2 μM and 20× is 4 μM. The final concentration of DSC1 is 0.2 μM.

FIG. 15A depicts the average Ct values obtained by using each enzyme combination for each copy level. At the 10-copy level Taq-B without the DSC molecules has no detectable Ct value. qPCR amplification performed in the presence of the DSC molecule has a measurable Ct value comparable to the one obtained with the chemically modified Taq. The Ct value also decreases with increasing DSC concentration. A greater PCR efficiency is achieved with increased DSC concentration.

FIG. 15B illustrates the amplification curves for each Taq-B DSC combination along with Taq-B and FastStart alone. Higher amplitudes are achieved with increased DSC concentration. The Taq-B DSC5 formulation at 10× exceeds the performance of the chemically modified Taq.

FIG. 15C shows the final amplitude for each combination at each copy level. Overall, the best performance is achieved in the presence of DSC5 molecule at the 10× concentration.

The phrase “consists essentially of” or “consisting essentially of” refers to elements in the claimed invention that are essential or needed for the claimed invention to work or operate in any embodiment described herein. For example, the blocking double stranded nucleic acid complex of the present invention, in an embodiment, consists essentially of the double stranded nucleic acid complex and the blocking molecule, both as described herein. Similarly, compositions, methods, kits or systems of the present invention consist essentially of the DSC described herein, along with DNA polymerase, buffers, a supply of adenine, guanine, cytosine and thymine, and primers, also as described herein.

### EXEMPLIFICATION

### Example 1

A Taq-B polymerase catalyzed PCR was performed using a system that amplifies a 1.1 kb region of pUC19 plasmid (FIGS. 1 and 2). PCR amplification was carried out using primers, with and without 3′OH modification. One primer set included 5′-AACAATTTCACACAGGAACAGCT-3′(SEQ ID NO: 21) and 5′-GTTTTCCCAGTCACGACGT-3′ (SEQ ID NO: 22) that have free 3′OH group. In the second primer set the availability of the 3′OH group was blocked by an inverted dT modification, 5′AACAATTTCACACAGCAACAGC/inverted T/−3′(SEQ ID NO: 23) and 5′-GTTTTCCCAGTCACGACG/inverted T/−3′(SEQ ID NO: 24). Reactions contained either 1×PCR buffer I (50 mM KCl, 1.5 mM MgCl₂, 20 mM Tris-HCl, pH 8.6 at 25° C.) or 1×PCR buffer II (10 mM (NH₄)₂SO₄, 10 mM KCl, 2 mM MgSO₄, 0.01% Triton X-100, 50% Glycerol, 20 mM Tris-HCl, pH 8.8 at 25° C.). Each reaction contained 0.2 mM dNTPs, 0.2 μM of each primer, 4 ng of pUC19 DNA, and 5 U of Taq-B polymerase. All PCR reactions were carried out in 100 μL volume on 2720 PCR Thermal Cycler (Applied Biosystems). Reaction conditions were as follows: initial denaturation at 95° C. for 3 min, followed by 35 cycles of 95° C. for 20 s, 55° C. for 20 s, 68° C. for 1 min 15 s, final extension at 68° C. for 7 min. After PCR, 20 μL of each sample was loaded on 1% agarose gel and visualized under UV light with an Alpha Innotech Corporation AlphaImager HP, 2401 Merced St. San Leandro, Calif. 94577 USA. Taq-B Polymerase, part number P725 L, is available from Enzymatics, Inc. 100 Cummings Center, Suite 336H, Beverly, Mass. 01915 USA. The DNA standard marker was the 1 kb DNA ladder, catalog #N3232 S available from New England Biolabs, 240 County Road Ipswich, Mass. 01938 USA.

### Example 2

PCR amplification protocol used in experiments depicted in FIG. 3-5 included 1×PCR buffer I (50 mM KCl, 1.5 mM MgCl₂, 20 mM Tris-HCl, pH 8.6 at 25° C.), 0.2 mM dNTPs, and 5 U of Taq polymerase in 100 μL reaction volume. PCR amplification was carried out using 0.2 μM of each forward 5′AAGGAGCTGGCTGACATTTTCG-3′ (SEQ ID NO: 25) and reverse 5′CGGGATATCGACATTTCTGCACC-3′ (SEQ ID NO: 26) primers that amplify a 1.9 kb region from Lambda phage DNA at 10,000 copies in the presence of 1 ng of E. coli genomic DNA as a competing foreign DNA. PCR experiments were performed on an Applied Biosystems 2720 thermal cycler. Reaction condition were initial denaturation at 95° C. for 5 min, followed by 40 cycles of 95° C. for 40 s, 56° C. for 30 s, 72° C. for 2 min, final extension at 72° C. for 7 min. After PCR, 20 μL of each sample was loaded on 1% agarose gel and visualized under UV light with an Alpha Innotech Corporation AlphaImager HP. The commercially available hot start DNA polymerases used include Amplitaq Gold, part number N8080246 available from Applied Biosystems, a division of Life Technologies Corp. 5791 Van Allen Way PO Box 6482, Carlsbad, Calif. 92008 USA. FastStart Taq DNA polymerase, catalog Number 12032902001, available from Roche Diagnostics Corporation, P.O. Box 50414, 9115 Hague Road, Indianapolis, Ind. 46250-0414 USA. The Lambda PCR protocol was adapted from Koukhareva and Lebedev (2009) Anal. Chem. 81:12 and is incorporated by reference in its entirety.

### Example 3

In FIG. 6 a 653-bp fragment of the β-actin gene from human placental DNA was amplified. All 100 μL PCR reactions contained 1× PCR buffer I (50 mM KCl, 1.5 mM MgCl₂, 20 mM Tris-HCl, pH 8.6 at 25° C.), 0.2 mM dNTPs, 0.5 μM of each forward 5′AGAGATGGCCACGGCTGCTT-3′ (SEQ ID NO: 26) and reverse 5′-ATTTGCGGTGGACGATGGAG-3′ (SEQ ID NO: 26) primers, 100 ng of template, and 5 U of Taq polymerase. Thermal cycling conditions were initial denaturation at 94° C. for 2 min, followed by 35 cycles of 94° C. for 30 s, 60° C. for 30 s, 72° C. for 45 s, final extension at 72° C. for 7 min. After PCR, 20 μL of each sample was loaded on 1% agarose gel and visualized under UV light with an Alpha Innotech Corporation AlphaImager HP. The PCR protocol for β-actin was adopted from Lebedev et. al. (2008) Nucleic Acid Research 31:20 which is incorporated by reference.

### Example 4

The PCR amplification protocol used in experiments depicted in a FIG. 7-11 included 1× qPCR buffer, 0.4 mM dNTPs, and 2.5 U of Taq polymerase in 25 μL reaction volume. PCR amplification was carried out using 1× of each oligo mix, A-FAM, B-HEX, and C-CY5 that amplify a 100-120-bp fragment of DNA A, B, and C, respectively at 1000 copies to 5 copies. DNA target series dilutions were prepared in qPCR reaction buffer. Assay mix, containing dNTPs, trehalose, Taq enzyme and oligo mix were prepared first in 5 μL final volume to which 20 μL of target mix was added. PCR experiments were performed on an Applied Biosystems 2720 thermal cycler. Reaction condition were initial denaturation at 95° C. for 10 min, followed by 40 cycles of 95° C. for 30 s, 56° C. for 1 min. After PCR, 20 μL of each sample was loaded on 3% agarose gel and visualized under UV light with an Alpha Innotech Corporation AlphaImager HP. The DNA standard marker was the 100 bp DNA ladder, catalog #N3231 S available from New England Biolabs, 240 County Road Ipswich, Mass. 01938 USA.

### Real Time PCR Experiments with TaqMan® Probe Detection

### Example 5

The PCR amplification used in experiments depicted in a FIG. 12-15 included 1× qPCR buffer, 0.4 mM dNTPs, and 2.5 U of Taq polymerase in 25 μL reaction volume (25 and 50 μL reaction volume in FIG. 14B). PCR amplification was carried out using 1× of each oligo mix, A-FAM, B-HEX, C-CY5, and HBB2 that amplify a 100-120-bp fragment of DNA A, B, C, and human placental DNA respectively at 1280 copies to 5 copies. DNA target series dilutions were prepared in qPCR reaction buffer. Assay mix, containing dNTPs, trehalose, Taq enzyme and oligo mix were prepared first in 5 μL final volumes to which 20 μL of target mix was added. Reaction condition were initial denaturation at 95° C. for 10 min, followed by 40 cycles of 95° C. for 30 s, 56° C. for 1 min.

The relevant teachings of all the references, patents and/or patent applications cited herein are incorporated herein by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A blocking double stranded nucleic acid complex for use in nucleic acid amplification; wherein the complex comprises:
a. an isolated double stranded nucleic acid molecule that comprises:
i. a first nucleic acid strand having a first sequence comprising between about 9 to about 40 nucleic acid bases, and
ii. a nucleic acid second strand having a second sequence comprising between about 9 to about 40 nucleic acid bases that are complementary to the first sequence, wherein the first nucleic acid strand and the second nucleic acid strand each having a 3′ end and a 5′ end,
wherein the double stranded nucleic acid molecule has a percentage of cytosine (C) and guanine (G) in a range between about 50% and about 70%; and
b. a blocking molecule, wherein the blocking molecule is covalently bonded to the 3′ end or the 5′ end of the first nucleic acid strand, the second nucleic acid strand, or both.

2. The blocking double stranded nucleic acid complex of claim 1, wherein the double stranded nucleic acid molecule has a melting temperature in a range between about 25° C. and about 90° C.

3. The blocking double stranded nucleic acid complex of claim 1, wherein the complex comprises DNA, or RNA.

4. The blocking double stranded nucleic acid complex of claim 1, wherein the blocking molecule consists from the group consisting of: deoxythymidine, dideoxynucleotides, 3′ phosphorylation, hexanediol, spacer molecules, 1′2′-dideoxyribose, 2′-0-Methyl RNA, and Locked Nucleic Acids (LNAs).

5. The blocking double stranded nucleic acid complex of claim 1, wherein the blocking molecule comprises:

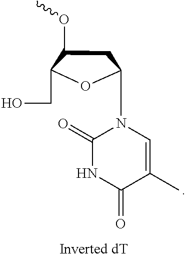

6. The blocking double stranded nucleic acid complex of claim 1, wherein the first sequence or the second sequence further comprises one or more uracil bases.

7. A blocking double stranded nucleic acid complex for use in nucleic acid amplification; wherein the complex comprises:
a. an isolated double stranded nucleic acid molecule that comprises:
i. a first nucleic acid strand having a first sequence comprising between about 9 to about 40 nucleic acid bases, and
ii. a nucleic acid second strand having a second sequence comprising between about 9 to about 40 nucleic acid bases that are complementary to the first sequence, wherein the first nucleic acid strand and the second nucleic acid strand each having a 3′ end and a 5′ end,
wherein the double stranded nucleic acid molecule has a melting temperature in a range between about 25° C. and about 90° C.;
b. a blocking molecule, wherein the blocking molecules is covalently bonded to the 3′ end or the 5′ end of the first nucleic acid strand, the second nucleic acid strand, or both.

8. The blocking double stranded nucleic acid complex of claim 7, wherein the blocking molecule consists from the group consisting of: deoxythymidine, dideoxynucleotides, 3′ phosphorylation, hexanediol, spacer molecules, 1′2′-dideoxyribose, 2′-0-Methyl RNA, and LNAs.

9. A blocking double stranded nucleic acid complex for use in nucleic acid amplification; wherein the complex comprises:
a. an isolated double stranded nucleic acid molecule that comprises:
i. a first nucleic acid strand having a first nucleic acid sequence greater than or equal to about 70% identity with a sequence comprising:
a. SEQ ID NO: 1, 2, 3, 4, 5, 6, 7, 8, 9, 10, 11, 12, 13, 14, 15, 16, or combination thereof;
b. a complement of SEQ ID NO: 1, 2, 3, 4, 5, 6, 7, 8, 9, 10, 11, 12, 13, 14, 15, 16, or combination thereof; or
c. a sequence that hybridizes to SEQ ID NO: 1, 2, 3, 4, 5, 6, 7, 8, 9, 10, 11, 12, 13, 14, 15, 16, or combination thereof; and
ii. a second nucleic acid strand having a second sequence comprising between about 9 to about 40 nucleic acid bases that are complementary to the first nucleic acid sequence, wherein the first nucleic acid strand and the second nucleic acid strand each having a 3′ end and a 5′ end,
wherein the double stranded nucleic acid molecule has a melting temperature in a range between about 25° C. and about 90° C.;
b. a blocking molecule, wherein the blocking molecules is covalently bonded to the 3′ end or the 5′ end of the first nucleic acid strand, the second nucleic acid strand, or both.

10. The blocking double stranded nucleic acid complex of claim 9, wherein the 3′ end of the first and second nucleic acid strand comprises the blocking molecule, wherein when the blocking double stranded nucleic acid complex interacts with a nucleic acid polymerase, the non-specific amplification products are thereby reduced.

11. The blocking double stranded nucleic acid complex of claim 9, wherein the complex has a melting temperature of about 48.9° C.

12. The blocking double stranded nucleic acid complex of claim 9, wherein the first sequence or the second sequence further comprises one or more uracil bases.

13. A composition for nucleic acid amplification; the composition comprises:
a. a buffer;
b. the blocking double stranded nucleic acid complex of claim 1, and
c. a thermostable polymerase.

14. The composition for nucleic acid amplification of claim 13, wherein the polymerase is a DNA polymerase consisting of the group: Taq DNA polymerase; BST DNA Polymerase; PFU DNA polymerase; Klenow DNA polymerase; T7 DNA polymerase; T4 DNA polymerase; Phi29 DNA polymerase; and RB69 DNA polymerase.

15. A composition for nucleic acid amplification of claim 13, wherein the range of concentration is between about 2 μM nucleic acid complex to every 5,000 U/mL of polymerase and 2 mM nucleic acid complex for every 5,000 U/mL of polymerase.

16. The composition for nucleic acid amplification of claim 13, wherein the buffer comprises a TRIS buffer, MOPS, or a HEPES buffer.

17. A method of amplifying a target nucleic acid molecule, the method comprises:
contacting the target nucleic acid molecule with a DNA polymerase and the double stranded nucleic acid complex of claim 1, wherein the double stranded nucleic acid complex binds to the DNA polymerase, at a temperature, ranging from about 25° C. to about 90° C.;
wherein amplified target nucleic acid molecules are obtained, and production of one or more non-specific amplification products or secondary products is reduced as compared to that not contacted with the double stranded nucleic acid complex.

18. The method of claim 17, wherein polymerase activity at a temperature between about 20° C. and 25° C. is reduced as compared to polymerase activity for a target nucleic acid molecule not contacted with the double stranded nucleic acid complex.

19. The method of claim 18, wherein polymerase activity at a temperature between about 20° C. and 25° C. is reduced in a range between about 50% and about 90%.

20. The method of claim 17, wherein an amount of amplified target nucleic acid molecules is increased, as compared to an amount of target nucleic acid molecules obtained when not contacted with the double stranded nucleic acid complex.

21. The method of claim 20, wherein the amount of amplified target nucleic acid obtained is increased in a range between about 2× and about 20×.

22. A method of amplifying a target nucleic acid molecule, the method comprises:
contacting the target nucleic acid molecule with a DNA polymerase from a species of an Archaebacteria and the double stranded nucleic acid complex of claim 6, wherein the double stranded nucleic acid complex binds to the DNA polymerase, at a temperature, ranging from about 25° C. to about 90° C.;
wherein amplified target nucleic acid molecules are obtained, and production of one or more non-specific amplification products or secondary products is reduced as compared to that not contacted with the double stranded nucleic acid complex.

23. The method of claim 22, wherein polymerase activity at a temperature between about 20° C. and 25° C. is reduced as compared to polymerase activity for a target nucleic acid molecule not contacted with the double stranded nucleic acid complex.

24. The method of claim 23, wherein polymerase activity at a temperature between about 20° C. and 25° C. is reduced in a range between about 50% and about 90%.

25. A method of amplifying a target nucleic acid molecule, the method comprises:
a. mixing a buffer, the target nucleic acid molecule, one or more primers, a DNA polymerase, a supply of adenine, guanine, cytosine and thymine, and the double stranded nucleic acid complex of claim 1;
b. allowing for amplification of the target nucleic acid molecule by increasing the temperature in one or more cycles, wherein the temperature ranges between about 25° C. to about 90° C., wherein amplified target nucleic acid molecules are obtained, and the production of one or more non-specific amplification products or secondary products is reduced as compared to that not contacted with the double stranded nucleic acid complex.

26. The method of claim 25, wherein polymerase activity at a temperature between about 20° C. and 25° C. is reduced as compared to polymerase activity for a target nucleic acid molecule not contacted with the double stranded nucleic acid complex.

27. The method of claim 25, wherein an amount of amplified target nucleic acid molecules is increased, as compared to target nucleic acid molecules not contacted with the double stranded nucleic acid complex.

28. A kit for nucleic acid amplification; the kit comprises:
a. the blocking double stranded nucleic acid complex of claim 1, and
b. a polymerase.

29. The kit of claim 28, wherein the polymerase is a DNA polymerase consisting of
the group: Taq DNA polymerase; BST DNA Polymerase; PFU DNA polymerase; Klenow DNA polymerase; T7 DNA polymerase; T4 DNA polymerase; Phi29 DNA polymerase; and RB69 DNA polymerase.
